(19) United States
(12) Patent Application Publication
(10) Pub. No.: US 2005/0182386 A1
(43) Pub. Date: Aug. 18, 2005

(54) Catheter with stiffening element

(76) Inventor: Steen Aggerholm, St. Heddinge (DK)

Correspondence Address: BRINKS HOFER GILSON & LIONE/CHICAGO/COOK, PO BOX 10395, CHICAGO, IL, 60610, US

(21) Appl. No.: 11/056,930

(22) Filed: Feb. 11, 2005

Related U.S. Application Data
(60) Provisional application No. 60/545,237, filed on Feb. 17, 2004.

(51) Int. Cl. A61M 25/00

(52) U.S. Cl. 604/524

(57) ABSTRACT

Catheters having a stiffening element are disclosed. The resultant catheters may include one or more access hubs. During use of the catheters, a stiffening element can provide an increased and variable resistance to deformation of the catheter body. The proximal end of the stiffening element may be coupled to the catheter hub. The stiffening element may move in the lumen it occupies as the catheter is threaded along the wire guide, thus allowing the catheter components to attain their lowest energy configuration at each bend.

This application claims priority to U.S. Provisional Application No. 60/545,237, filed Feb. 17, 2004, which is hereby incorporated by reference herein.

### BACKGROUND

Catheters are tube-like medical instruments that may be inserted into a body cavity, organ, or blood vessel for diagnostic or therapeutic reasons. Catheters may be designed for insertion into the vasculature and are available for a wide variety of purposes, including diagnosis, interventional therapy, drug delivery, drainage, perfusion, and the like. They also may be useful for other procedures, such as gynecological procedures, cardiac procedures, general interventional radiology procedures, and the like. Catheters for each of these and other purposes can be introduced to numerous target sites within a patient's body by guiding the catheter through an incision made in the patient's skin and a blood vessel and then through the body to the target site.

Catheters generally have an elongated, flexible catheter body with a catheter side wall enclosing one or more catheter lumens. The lumens can extend from a catheter body proximal end, where the catheter body is coupled to a relatively more rigid catheter hub, to a distal end. The one or more lumens may have the same diameter throughout the length of the catheter or they may taper, such as when the lumens have a larger diameter at the proximal end than at the distal end. The catheter hub typically has one or more access hubs that provide for the insertion of wire guides or the attachment of syringes or other devices, for example. The catheter body may be relatively straight, inherently curved, or curved by insertion of a stiffening element or wire guide. The catheter body may assume a straight or linear configuration, when free from external bending forces.

The catheter body may be highly flexible, thus able to pass through the tortuous twists and turns of a patient's v asculature. In some cases, the catheter body may have a shaped distal end portion including curves and bends that are selected to facilitate introduction and placement of the catheter in the vascular system. A particular geometry of curves and/or bends may be selected to accommodate the intended use of the catheter. The distal end of the catheter also may be equipped with an inflatable balloon to expand a medical device, such as a stent, and/or to dilate a vessel.

The process of guiding a catheter through a vessel becomes very difficult when the vessel becomes small and when the target site is deep within the vascular. Most small blood vessels must be accessed through highly tortuous pathways. In order to reach a target vessel, the catheter may need to be quite flexible in order to follow the tortuous path, while at the same time stiff enough to allow the distal end of the catheter to be manipulated from it proximal end.

The end of the catheter that remains external to the body cavity (proximal end) generally terminates in a catheter hub. Catheter hubs may include one or more lumen access hubs. The lumen access hubs provide ingress and egress from the mouths of the access hubs to one or more lumens within the catheter body, such as a central or first lumen and an outer or second lumen. The lumens running longitudinally through the catheter body generally have a substantially smaller diameter than the access hubs. The access hubs may include female luer type connectors or another type of connector.

Fluids, gases, wires, and the like may be passed from the mouths of the access hubs, through the lumens, and optionally into the body cavity. For example, a wire guide may be placed at the desired location in the body cavity. The proximal end of the first catheter lumen then may be threaded over the wire guide until the wire guide exits the first lumen access hub. In this manner, the wire guide may be utilized to guide the catheter to the desired location in the body cavity. A fluid, such as a viscous liquid, pharmaceutical preparation, or gas, may be directed through the second lumen access hub and into the second lumen. If the catheter is a balloon type catheter, this fluid may inflate a balloon at the distal end of the catheter that is in fluid communication with the second lumen.

The bodies and side walls of catheters may be fabricated and dimensioned to minimize the outer diameter of the catheter body and the thickness of the side wall. In this fashion, the diameter of the catheter lumens may be maximized while retaining sufficient side wall flexibility and strength characteristics to enable the catheter to be used for the intended medical purpose.

When a catheter is tracked over a wire guide, the progress or “pushab ility” of the catheter may be impeded by kinking and columnar collapse of the catheter onto the wire guide. This problem is especially acute when the material from which the catheter is made is highly flexible to allow the catheter to pass over the difficult curves of the wire guide when the wire guide is positioned in a tortuous path. Thus, the more flexible the material from which the catheter body is made, the more easily it may follow the wire guide; however, the more likely the catheter is to bind with the wire guide when a pushing force is applied to the proximal end of the catheter body. In this way, the catheter design should provide a balance between the flexibility required to allow passage of the catheter through the sharp bends of increasingly narrow vessels, yet allow sufficient longitudinal stiffness to provide for the desired placement of the distal portion of the catheter.

### BRIEF SUMMARY

A catheter is provided that includes a stiffening element placed in a lumen of the catheter. The catheter may include an elongate body having a plurality of lumens, such as a first lumen and a second lumen. The first lumen may be defined by an inner tube, while the second lumen may be defined by the space between the inner surface of an outer tube and the outer surface of the inner tube. A hub may be coupled to the proximal end of the catheter body having first and second access hubs in fluid communication with the first and second lumens, respectively. The stiffening element may run longitudinally in one of the lumens and be coupled to the hub and a proximal end of the inner tube. The stiffening element can enhance the resistance of the catheter body to compression along its longitudinal axis.

Other features, methods, and advantages of the invention will be, or will become, apparent to one with skill in the art upon examination of the following figures and detailed description. It is intended that all such additional features, methods, and advantages are included within this description, are within the scope of the invention, and are protected by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention.

FIG. 1 depicts an axial cross-sectional view of an elongate, flexible catheter.

FIG. 2 is a longitudinal cross-sectional view of a catheter body coupled to a catheter hub.

FIG. 3 is an axial cross-section taken through line X-X of FIG. 2 depicting the coupling of an inner tube and a stiffening element to the proximal portion of a catheter hub.

FIG. 4 is an axial cross-section taken through line Z-Z of FIG. 2 depicting the coupling of an outer tube to the distal portion of a catheter hub.

### DETAILED DESCRIPTION

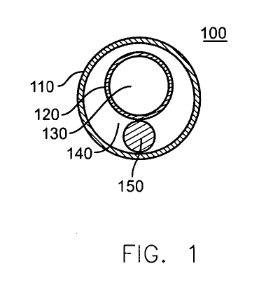

FIG. 1 depicts an axial cross-sectional view of an elongate, flexible catheter body 100 having a longitudinal axis extending between a proximal end and a distal end. The catheter body 100 may include a plurality of lumens, such as an inner lumen 130 and an outer lumen 140, defined by a plurality of tubes, such as outer tube 110 and inner tube 120, for example, and a stiffening element 150. The catheter body 100 may have a length in the range from about 40 centimeters (cm.) to about 200 cm., usually having a length in the range from about 60 cm. to about 175 cm. The diameter of the catheter body 100 may be in the range of about 0.67 millimeters (mm.) (2 F) to about 7 mm. (21 F).

The outer tube 110 may terminate proximal of the inner tube 120, thus being shorter in length. The outer tube 110 may have an external diameter of about 0.67 mm. to about 7 mm., while the inner tube 120 may have an external diameter of about 0.33 mm. to about 6 mm. The outer tube 110 also may have an external diameter of about 1.3 mm., while the inner tube 120 may have an external diameter of about 0.74 mm.

The outer tube 110 may contain one or more inner tubes defining additional inner lumens. In this fashion, a first lumen may be formed in the interior of an inner tube, while a second lumen may be formed between an outer wall of an inner tube and the inner wall of the outer tube. When an inner tube resides within the outer tube, the tubes substantially may share the same center, thus being substantially coaxial. The inner lumens may have inside diameters ranging from about 0.25 mm. to about 5.67 mm.

The body catheter body 100 may be made from any suitable material, including, but not limited to, polyethers and polyester block amides. For example, the outer tube may be made from a polyether block amide, which may include a copolymer of amide monomers copolymerized with polyether monomers. Because the amide monomers may have greater structural “rigi dity” in comparison to the polyether monomers, the resistance of the resulting catheter body to deformation, such as bending or stretching, may be altered. One example of a suitable polyether block amide from which the catheter body and/or side wall may be made is PEBAX®, which is available from Elf Atofina, Philadelphia, Pa. In one aspect, a blend of PEBAX® polyether block amides may be used.

The inner tube or tubes that define the lumens may be made from a single material, such as a lubricious polymer, or a combination of materials. Lubricious polymers include, but are not limited to, fluorocarbons, such as polytetrafluoroethylene (PTFE), polyamides, such as nylons, polyether block amides (PEBA), polyolefins, polyimides, and the like. The inner tube also may be a laminate structure that includes a non-lubricious outer layer and a more lubricious inner layer or coating.

Unlike the wire guide, which is utilized to position the catheter in the patient during use, the stiffening element 150 may not be removed from the catheter. The stiffening element 150 can alter the resistance of the catheter body to deformation, such as to the bending forces previously described and to longitudinal compression forces that tend to collapse the catheter body onto the wire guide during use. In this manner, the stiffening element 150 can provide improved resistance to kinking and thus enhanced “pushability” to the catheter body 100 as it is tracked over a wire guide during use. Thus, the material from which the catheter is made can be highly flexible, thereby allowing the catheter to follow the wire guide, while the stiffening element reduces the tendency of the catheter body to bind with the wire guide when a pushing force is applied to the proximal end of the catheter body. In this way, the combination of the catheter body and stiffening element can provide a desirable balance between flexibility and longitudinal stiffness, thus allowing the distal portion of the catheter to be more easily placed at the desired location during use.

The stiffening element 150 may run longitudinally in at least one of the plurality of lumens. The stiffening element 150 may run through the outer lumen 140. The stiffening element may displace the inner tube 120 from the actual center of the outer tube 110 (as depicted in FIG. 1), while the inner lumen 130 remains within the outer lumen 140. The stiffening element 150 may be in contact with the outer tube 110 and the inner tube 120 for a portion of or substantially all of its length. Radiopaque markers (not shown) also may be placed at any location on the catheter body, for example under a dilation balloon (not shown), if the catheter body is so equipped. The catheter 100 may have other configurations including those with fewer or additional components as is known in the art.

The stiffening element 150 may be solid or hollow and may be made from any material that provides the desired resistance to deformation. In one aspect, stiffening element 150 may be made from nitinol, spring steel, stainless steel, a metal alloy, carbon fiber, or plastic. In another aspect, the stiffening element 150 may be a solid metal wire.

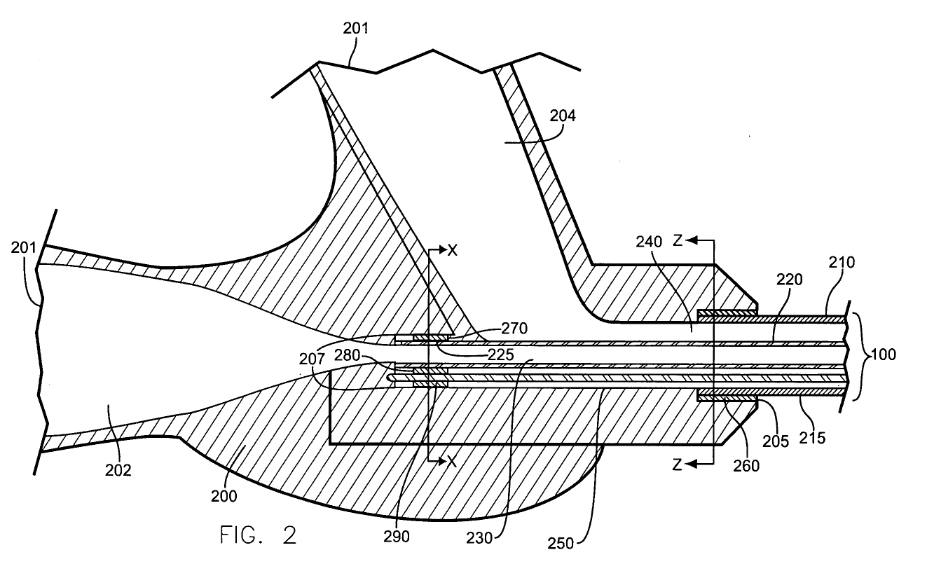

The stiffening element 150 may be free from attachment to the catheter body 100. In one aspect, the stiffening element 150 only may be coupled to a hub, such as the hub 200 discussed below regarding FIG. 2. Thus, as the catheter is threaded over the wire guide during use, the catheter components including the outer tube 110, the inner tube 120, and the stiffening element 150 may attain their lowest energy configuration with respect to each other at each bend in the wire guide. Because the stiffening element 150 can move laterally in its lumen, such as the second lumen 140, with respect to the outer and inner tubes, 110 and 120, the catheter 100 can retain the lateral flexibility to follow to the wire guide, while providing sufficient longitudinal stiffness to resist compression along its longitudinal axis. Furthermore, by coupling the stiffening element 150 only to the hub 200, a catheter that is easier to manufacture may be provided.

The stiffening element 150 may have a substantially constant outside diameter from its proximal to its distal end, or it may taper toward its distal end. The stiffening element 150 may begin to taper about 15 cm. proximal to its distal end. The stiffening element 150 may taper from an outside diameter of about 0.3 mm. at its proximal end to a terminal diameter of about 0.075 mm. at its distal end.

The stiffening element 150 may extend through only a portion of the catheter body 100 or through the complete length of the catheter body 100. When the catheter body 100 is equipped with a distal balloon (not shown), the distal end of the stiffening element 150 may terminate from about 5 to about 20 cm. proximal from the proximal end of the balloon. The stiffening element 150 also may terminate about 10 cm. or more than 20 cm. proximal from the proximal end of the balloon.

The proximal end of the balloon may be coupled to the distal terminus of the outer tube 110, while the distal end of the balloon may be coupled to the distal terminus of the inner tube 120. In another aspect, the distal end of the inner tube 120 may extend distally of the distal end of the balloon. In either aspect, fluid communication may be provided between the interior of the balloon and the outer lumen 140. The balloon also may be coupled at its proximal and distal ends to the outer tube 110 with one or more passageways through the outer tube providing fluid communication with the outer lumen 140. Additional configurations may be used to establish fluid communication between the outer lumen 140 and the balloon interior.

FIG. 2 is a longitudinal cross-sectional view of the catheter body 100 coupled to a catheter hub 200. The catheter hub 200 has distal and proximal portions, with the distal portion being closer to the catheter body 100 than the proximal portion. The catheter hub 200 may include one or more access hubs, such as a first lumen access hub 202 and a second lumen access hub 204. The lumen access hubs may provide ingress and egress from the mouths 201 of the access hubs to one or more lumens, such as the first lumen 230 and the second lumen 240. The lumens may have a substantially smaller diameter than the access hubs. The first lumen access hub 202 may be in fluid communication with the first lumen 230, while the second lumen access hub 204 may be in fluid communication with the second lumen 240. The catheter hub 200 may have other configurations including those with fewer or additional components.

The access hubs 202 and 204 may include female luer type connectors or another type of connector. The access hubs may be made from a plastic that allows the user to see air bubbles that may exist in a contained fluid. Thus, one or more of the access hubs may have clarity sufficient for air bubbles to be observed. This clarity can provide a significant benefit because the user can monitor fluid introduction, such as during the inflation of a balloon, for undesirable air bubbles.

The outer tube 210 of the catheter body 100 may be coupled to a distal portion of the catheter hub 200 by an adhesive residing in a region 260 between a distal inner surface 205 of the catheter hub 200 and an outer surface 215 of the outer tube 210. In this manner, the outer tube 210 may only be coupled to the hub. By locating the adhesive in the region 260 between the inner surface 205 of the catheter hub 200 and the outer surface 215 of the outer tube 210, an inner tube 220 and a stiffening element 250 may not be coupled to the distal portion of the catheter hub 200. Beneficially, by limiting coupling to the outer surface 215 of the outer tube 210, the inflation lumen may be substantially free of occlusion by the adhesive.

The inner tube 220 of the catheter body 100 may be coupled to a proximal portion of the catheter hub 200 by an adhesive residing in a region 270 between a proximal inner surface 207 of the catheter hub 200 and an outer surface 225 of the inner tube 220. An outer surface 225 of the inner tube 220 also may be coupled to the stiffening element 250 by an adhesive residing in a region 280 between the outer surface 225 of the inner tube 220 and the stiffening element 250. In this manner, the inner tube 220 may be coupled to the catheter hub 200, but not to the outer tube 210. The stiffening element 250 also may be coupled to the proximal portion of the catheter hub 200 by an adhesive residing in a region 290 between the proximal inner surface 207 of the catheter hub 200 and the stiffening element 250. In this fashion, the inner tube 220 and the stiffening element 250 may be coupled to each other and to a proximal portion of the catheter hub 200, but not to the outer tube 210. This coupling of the inner tube 220 and the stiffening element 250 may occur at substantially the same proximal region of the catheter hub 200. The stiffening element 250 may terminate distal to the proximal termination of inner tube 220 or at substantially the same region.

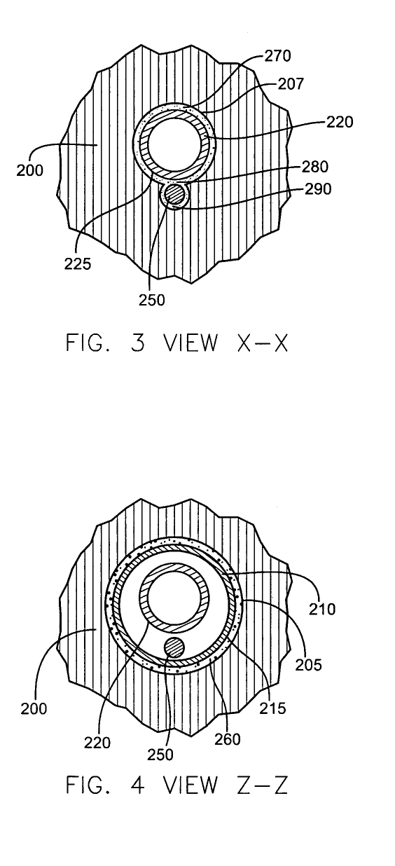

FIG. 3 is an axial cross-section taken through line X-X of FIG. 2. FIG. 3 depicts in greater detail the coupling of the inner tube 220 and the stiffening element 250 to the proximal portion of the catheter hub 200. An outer surface 225 of the inner tube 220 may be coupled to a proximal inner surface 207 of the catheter hub 200. An adhesive may be present in region 270 between the proximal inner surface 207 of the hub and the outer surface 225 of the inner tube 220, in region 280 between the outer surface 225 of the inner tube 220 and the stiffening element 250, and in region 290 between the stiffening element 250 and the proximal inner surface 207 of the hub. While regions 270, 280, and 290 are shown as being one continuous region, which contains an adhesive, one or more of the regions may be distinct. Additional arrangements may be utilized to couple the inner tube 220 and the stiffening element 250 to the proximal portion of the catheter hub 200.

FIG. 4 is an axial cross-section taken through line Z-Z of FIG. 2. FIG. 4 depicts in greater detail the coupling of the outer tube 210 to the distal portion of the catheter hub 200. An outer surface 215 of the outer tube 210 may be coupled to a distal inner surface 205 of the catheter hub 200. An adhesive may be present in region 260 between the distal inner surface 205 of the hub and the outer surface 215 of the outer tube 210. While region 260 is shown as one continuous region, which may contain an adhesive, the region may not be continuous. Additional arrangements may be utilized to couple the outer tube 210 to the distal portion of the catheter hub 200. In this aspect, inner tube 220 and stiffening element 250 are not coupled to the distal portion of the catheter hub 200.

The coupling between the hub, such as the hub 200 of FIG. 2, the tubes, and the stiffening member may be provided by any method that holds the various catheter components together during use. An adhesive may be used. The adhesive may be any bonding agent, such as a thermoplastic or resin, which holds the catheter components together. The adhesive may include a curable resin that is applied to the catheter body as an uncured resin in a semi-solid, liquid, or powdered state. The uncured resin may then be cured with light, heat, radiation, radio frequency, air, a chemical accelerator, or other process that results in a hardening of the resin.

The catheter components also may be coupled without a resin, such as by heat fusing the hub material to the catheter body or by an overmold process as described in U.S. Provisional Patent Application Ser. No. 60/501,991, which is incorporated by reference in its entirety. An insert molding process also may be used to couple the components of the catheter. Additional coupling methods known to those of ordinary skill in the art also may be used.

While various embodiments of the invention have been described, it will be apparent to those of ordinary skill in the art that other embodiments and implementations are possible within the scope of the invention. Accordingly, the invention is not to be restricted except in light of the attached claims and their equivalents.

1. A catheter comprising:
an elongate flexible catheter body having a plurality of lumens including a first lumen defined by an inner tube and a second lumen defined by an outer tube exterior to the first lumen, the flexible catheter body having a longitudinal axis extending between a proximal end and a distal end;
a hub having a plurality of access hubs including a first access hub and a second access hub, the first access hub being in fluid communication with the first lumen and the second access hub being in fluid communication with the second lumen, where the outer tube is coupled to the hub and the inner tube is coupled to the hub; and
a stiffening element running longitudinally in the second lumen, where the stiffening element is coupled to the hub.

2. The catheter of claim 1, where the inner tube and the outer tube are substantially coaxial.

3. The catheter of claim 1, where the stiffening element displaces the inner tube from the actual center of the outer tube.

4. The catheter of claim 1, where at least a portion of the stiffening element contacts the inner tube and the outer tube.

5. The catheter of claim 1, where the outer tube has an external diameter of about 0.67 mm. to about 7 mm.

6. The catheter of claim 1, where the inner tube has an external diameter of about 0.33 mm. to about 6 mm.

7. The catheter of claim 1, where the stiffening element is a wire.

8. The catheter of claim 7, where the wire tapers toward the distal end of the catheter body.

9. The catheter of claim 8, where the wire begins to taper about 15 cm. proximal to its distal terminus.

10. The catheter of claim 8, where the taper is from an outside diameter of about 0.3 mm. to a terminal outside diameter of about 0.075 mm.

11. The catheter of claim 1, where the proximal and distal ends of the stiffening element do not substantially change its position with respect to the proximal and distal ends of the catheter body during use.

12. The catheter of claim 1, where a proximal end of the stiffening element terminates distal to a proximal end of the inner tube.

13. The catheter of claim 1, where the stiffening element is not coupled to the outer tube.

14. The catheter of claim 1, where the coupling of the outer tube to the hub is distal to the coupling of the inner tube to the hub.

15. The catheter of claim 1, where the coupling of the outer tube to the hub is distal to the coupling of the stiffening element to the hub.

16. The catheter of claim 1, where a proximal end of the stiffening element and the proximal end of the inner tube are coupled to the hub at substantially the same region of the hub.

17. The catheter of claim 1, where the stiffening element is coupled to a proximal end of the inner tube.

18. The catheter of claim 1, further comprising a balloon coupled near the distal end of the catheter body.

19. The catheter of claim 18, where a distal end of the stiffening element terminates from about 5 cm. to about 20 cm. proximal to the proximal end of the balloon.

20. The catheter of claim 18, where the outer tube terminates at a proximal end of the balloon.

21. The catheter of claim 18, where a distal end of the balloon is coupled to the inner tube.

22. The catheter of claim 18, further comprising at least one radiopaque marker on the catheter body.

23. The catheter of claim 1, where the lumens are defined by an outer tube and a plurality of inner tubes.

24. The catheter of claim 1, where the couplings are provided by at least one of a thermoplastic, a resin, and combinations thereof.

25. A method of making a catheter, comprising:
providing an elongate flexible catheter body having a plurality of lumens including a first lumen defined by an inner tube and a second lumen defined by an outer tube exterior to the first lumen, the flexible catheter body having a longitudinal axis extending between a proximal end and a distal end;
providing a hub having a plurality of access hubs including a first access hub and a second access hub;
providing a stiffening element running longitudinally in the second lumen;
coupling the outer tube to a distal region of the hub;
coupling the inner tube to a proximal region of the hub, where the first access hub is in fluid communication with the first lumen and the second access hub is in fluid communication with the second lumen; and
coupling the stiffening element to the proximal region of the hub.
